(19) United States
(12) Patent Application Publication
(10) Pub. No.: US 2007/0112181 A1
(43) Pub. Date: May 17, 2007

(54) Method for enrichment/seperation of protein or peptide

(76) Inventors: Makoto Watanabe, Osaka (JP); Eiichi Matsuo, Kyoto (JP); Chikako Toda, Kyoto (JP); Osamu Nishimura, Hyogo (JP)

Correspondence Address: RADER FISHMAN & GRAUER PLLC, LION BUILDING, 1233 20TH STREET N.W., SUITE 501, WASHINGTON, DC, 20036, US

(21) Appl. No.: 10/582,882

(22) PCT Filed: Dec. 21, 2004
(86) PCT No.: PCT/JP04/19677

§ 371 Date: Jun. 14, 2006

(30) Foreign Application Priority Data
Dec. 25, 2003 (JP) 2003-430898

(51) Int. Cl. C07K 1/00

(52) U.S. Cl. 530/412

(57) ABSTRACT

The present invention finds a media that can highly selectively retain proteins or peptides to be enriched/separated, as well as provides a method for selectively enriching/separating proteins or peptides using such a media. A method for enrichment/separation of a protein or a peptide, comprising separating a protein or a peptide containing an amino acid residue with a π electron-containing group by using a media with a π electron-containing group. Preferably, the amino acid residue is a tryptophan residue or a tryptophan residue modified with a sulfenyl compound, and the media is a media with phenyl group.

### TECHNICAL FIELD

The present invention relates to the field in proteomics or exhaustive analysis for protein.

### BACKGROUND ART

Peptide mass fingerprinting (PMF), a technique that combines 2D-PAGE with the benefit of mass spectrometer, has thus far been a main analytical technique used in the field of proteomics and exhaustive analysis of protein. Also, the differential display analysis using 2D-PAGE, has been used as a tool for determining relative quantification of proteins. These techniques have defects, however, in terms of the reproducibility of protein separation and gel staining, and the solubility of proteins. To address these problems, exhaustive analysis of protein that employs stable isotopes has been developed as an approach to next-generation protein analysis.

On the other hand, sulfenyl compounds are known as selective labeling reagents for tryptophan residues. Of these compounds, 2-nitrobenzene sulfenyl chloride (NBSCl) has been reported to act as a reagent that can selectively modify tryptophan residues in an acidic solution (Scoffone E., Fontana A., and Rocchi R., Biochemical Biophysical Research Communications, 1966, 25, 170; and Scoffone E., Fontana A., and Rocchi R. Biochemistry 1968, 7, 971).

In an experiment conducted by Hiroki Kuyama et al., two series of biological samples, normal and hyperglycemic, are separately derived from two lines of rats. Proteins in one sample are then labeled with a non-labeled NBSCl reagent at the tryptophan residues and proteins in the other sample are labeled with a ¹³C-labeled NBSCl reagent at the tryptophan residues. The two labeled samples are mixed and the mixture is subjected to-enzymatic digestion by trypsin. The peptides containing tryptophan residues are separated and are subjected to mass spectrometry. The peptides detected as paired peaks that have 6 daltons mass difference are then subjected to sequence analysis and the relative quantification of the peptides (Hiroki Kuyama, Makoto Watanabe, Chikako Toda, Eiji Ando, Koichi Tanaka, and Osamu Nishimura, Rapid Communications in Mass Spectrometry 2003, 17, 1642-1650). The separation of the tryptophan-containing peptides was performed by reversed-phase chromatography on an ODS column or Sephadex LH-20.

On the other hand, a media that includes phenyl groups as π electron-containing groups is described in 2001/2002 YMC GENERAL CATALOG HPLC COLUMN & GEL, p. 52 (YMC Corp.).

### DISCLOSURE OF THE INVENTION

### Object of the Invention

Accordingly, it is an objective of the present invention to find a media that can highly selectively retain proteins or peptides to be enriched/separated, as well as to provide a method for selectively enriching/separating proteins or peptides using such a media.

### SUMMARY OF THE INVENTION

In the course of their studies, the present inventors have discovered that the described objectives of the present invention can be achieved by applying the unique selectivity derived from the π-π electron interactions between π electron compounds to enrich/separate proteins or peptides. This discovery eventually led to the present invention. The invention thus is based on a principle that proteins or peptides containing π electron group-containing amino acid residues can be selectively separated by taking advantage of the π-π electron interactions between the π electron-containing media and the proteins or the peptides containing an amino acid residue with a π electron group.

The present invention thus comprises the following aspects:

(1) A method for enrichment/separation of a protein or a peptide, comprising separating a protein or a peptide containing an amino acid residue with a π electron-containing group by using a media with a π electron-containing group.

(2) The method according to (1), wherein the amino acid residue with a π electron-containing group is tryptophan residue.

(3) The method according to (1) or (2), wherein the π electron-containing group of the media is phenyl group.

(4) A method for enrichment/separation of a protein or a peptide, comprising separating a protein or a peptide containing an amino acid residue with a π electron-containing modifying group, which is modified with a π electron-containing compound, by using a media with a π electron-containing group.

(5) The method according to (4), wherein the amino acid residue is tryptophan residue.

(6) The method according to (4) or (5), wherein the π electron-containing compound is a sulfenyl compound having π electrons.

(7) The method according to (6), wherein the sulfenyl compound is 2-nitrobenzene sulfenyl chloride.

(8) The method according to any one of (4) to (7), wherein the π electron-containing group of the media is phenyl group.

(9) A method for enrichment/separation of a peptide, comprising the steps of:

fragmenting a protein or a peptide containing an amino acid residue with a π electron-containing group, to obtain a fragmented sample solution which contains a peptide fragment containing the amino acid residue with the π electron-containing group and a peptide fragment with no π electron-containing group; and

exposing the fragmented sample solution to a media with a π electron-containing group, to separate the peptide fragment containing the amino acid residue with the π electron-containing group from the peptide fragment with no π electron-containing group.

(10) A method for enrichment/separation of a peptide, comprising the steps of:

modifying a protein or a peptide with a π electron-containing compound to obtain a sample solution which contains a protein or a peptide containing an amino acid residue with a π electron-containing modifying group;

fragmenting the protein or the peptide containing the amino acid residue with the π electron-containing modifying group, to obtain a fragmented sample solution which contains a peptide fragment containing the amino acid residue with the π electron-containing group and a peptide fragment with no π electron groups; and

exposing the fragmented sample solution to a media with a π electron-containing group, to separate the peptide fragment containing the amino acid residue with the π electron-containing group from the peptide fragment with no π electron-containing group.

By employing a media that has a high ability to selectively retain proteins or peptides to be enriched/separated, the present invention makes it possible to enrich/separate such proteins or peptides in a highly selective manner. The method of the present invention enables more effective and accurate proteome analysis of various biological samples, including determination of relative quantification and sequence analysis of peptides or proteins in biological samples using a mass spectrometer.

### BRIEF DESCRIPTION OF THE DRAWINGS

FIG. 1 shows a MALDI-TOF MS spectrum of Example prior to enrichment of peptide fragments containing labeled tryptophan residues.

FIG. 2 shows MALDI-TOF MS spectra for the first fraction to the sixth fraction of twelve wash fractions collected in Example during washing with a wash buffer.

FIG. 3 shows MALDI-TOF MS spectra for the seventh fraction to the twelfth fraction of twelve wash fractions collected in Example during washing with the wash buffer.

FIG. 4 shows (a) MALDI-TOF spectra for the first fraction to the fifth fraction eluted in Example with an elute buffer, and (b) an enlarged view of one of the paired peaks indicated by arrows in (a).

### MODES FOR CARRYING OUT THE INVENTION

The present invention concerns a enrichment/separation technique for use in chromatography of proteins or peptides that takes advantage of the π-π electron interactions between π electron groups of amino acid residues in proteins or peptides to be enriched/separated and π electron groups of a media to serve as the stationary phase of the chromatography.

According to the present invention, proteins or peptides containing amino acid residues with π electron-containing groups can be enriched/separated in a selective manner. While the π electron-containing groups are not limited, aromatic hydrocarbon groups are preferred. According to the present invention, the amino acid residue with π electron-containing group is especially preferably tryptophan residue. Since tryptophan is one of the least abundant amino acid residues in proteins, the mass spectra obtained by the method of the present invention are simple and easy to interpret.

In the present invention, the amino acid residue with the π electron-containing group may be an amino acid residue of a protein or a peptide which is previously modified with a π electron compound. The π electron compound for modifying amino acid residues is preferably a sulfenyl compound. A sulfenyl compound can selectively modify tryptophan residues, preferred amino acid residues of the present invention. While the sulfenyl compound may be any sulfenyl compound that is represented by the general formula R—S—X (where R represents an organic group, and X represents a leaving group), it is preferred that the organic group R be an aromatic hydrocarbon group. Examples of the sulfenyl compound include 2-nitrobenzene sulfenyl chloride, 4-nitrobenzene sulfenyl chloride, 2,4-benzene sulfenyl chloride, and 2-nitro-4-carboxybenzene sulfenyl chloride. Of these, 2-nitrobenzene sulfenyl chloride shown by the following structural formula (I) is particularly preferred:

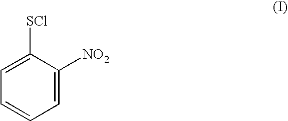

On the other hand, the media for separating the above-described protein or peptide containing the amino acid residues with π electron groups has a π electron-containing group. While the π electron-containing group of the media is not limited, aromatic hydrocarbon groups, such as phenyl group, are preferred. The use of such a media allows the separation that takes advantage not only of the hydrophobic interaction but also of the π-π electron interaction and is thus preferred. According to the present invention, the media is exposed to the above-described protein or peptide with π-electron groups. Specifically, a YMC-PackPh column (YMC Corp.) packed with the media with phenyl groups to serve as the solid phase may be used.

The present invention can be used in the exhaustive analysis of protein for various biological samples, including determination of relative quantification and sequence analysis of peptides or proteins using a mass spectrometer. In one example, two labeling reagents, a stable isotope-labeled form and an non-labeled form of the above-described sulfenyl compound, are prepared. Two series of biological samples (e.g., normal cells and cancer cells) are separately prepared. One of the samples is then performed tryptophan-labeling treatment with the stable isotope-labeled form of the above sulfenyl compound, and the other is performed tryptophan-labeling treatment with the non-labeled form of the same compound. The labeled samples are mixed with each other and the mixture is subjected to fragmentation. This results in a mixture containing labeled peptide fragments along with non-labeled peptide fragments.

Subsequently, the labeled peptide fragments are separated from the peptide fragment mixture, and this is where the present invention is preferably applied. Specifically, Using a media with for example phenyl groups, the labeled peptide fragments in the peptide fragment mixture are allowed to selectively adsorb onto the media, the other non-labeled peptide fragments are washed off, and the adsorbed labeled peptide fragments are subsequently eluted. In this manner, the labeled peptide fragments can selectively be separated. The separated labeled peptide contains two types of labeled peptides: one labeled with the stable isotope-labeled compound and the other labeled with the non-labeled compound. The two peptides are detected in mass spectrometry as paired peaks that have 6 daltons mass difference which corresponds to the mass difference between the two labeling reagents. The paired peaks also have an area ratio corresponding to the relative quantification of the two labeled peptides. The detected paired peak allows determination of the relative quantification of the peptides and sequence analysis.

When applied to actual biological samples, conventional Sephadex-based reversed-phase chromatography techniques often result in a deviation in the resolution and the reproducibility of the results. This drawback can be eliminated by the use of the method of the present invention. The method of the present invention, therefore, is more practical than conventional techniques and enables more effective and accurate protein analysis.

### EXAMPLES

The present invention will now be described with reference to examples, which are not intended to limit the scope of the invention in any way. Unless otherwise specified, the quantities indicated by ‘%’ are by weight.

Sera from Crj: Wistar rats and GK/Crj rats (Goto-Kakizaki) (each derived from Charles River Japan) were used as samples.

(a) Sample Treatment

50 μl of a Crj: Wistar rat serum was pretreated with albumin/globulin removing kit (Aurum Serum Protein Mini Kit, Bio-Rad). Proteins were quantitated by BCA method and a sample solution was prepared containing 100 μg of total proteins. 15 μl of this sample solution was added to an aqueous solution containing 0.1% SDS and 5 mM EDTA, and the mixture was boiled in boiled water for 5 min., followed by cooling in an ice bath.

Meanwhile, a NBSCl reagent (2-nitro[¹²C₆]benzene sulfenyl chloride, referred to as (¹²C) NBSCl, hereinafter) was prepared. 35 μl acetic acid solution dissolved 0.17 mg of (¹²C) NBSCl reagent therein was then added to the pretreated sample solution obtained above, and the mixture was incubate in a dark place at room temperature over night. This gave a ¹²C-labeled form of Crj: Wistar rat serum.

50 μl of serum derived from a GK/Crj (Goto-Kakizaki) rat was pretreated with albumin/globulin removing kit (Aurum Serum Protein Mini Kit, Bio-Rad). Proteins were quantitated by BCA method and a sample solution was prepared containing 100 μg of total proteins. 15 μl of this sample solution was added to a 0.1% aqueous SDS solution containing 5 mM EDTA, and the mixture was boiled in boiled water for 5 min., followed by cooling in an ice bath.

Meanwhile, a ¹³C stable isotope-labeled form of the above (12C) NBSCl reagent (2-nitro[¹³C₆]benzene sulfenyl chloride, referred to as (¹³C) NBSCl, hereinafter) was prepared. 35 μl acetic acid solution dissolved 0.17 mg of (¹²C) NBSCl reagent therein was then added to the pretreated sample solution obtained above, and the mixture was incubated in a dark place at room temperature over night. This gave a (¹³C)-labeled form of GK: Crj (Goto-Kakizaki) rat serum.

The two labeled forms thus obtained were mixed to make a sample mixture, which was then desalted with Sephadex LH-20 (Pharmacia) and was dried up in a centrifugal evaporator.

To the dried sample mixture, 44 μl of an aqueous solution of 0.01% SDS/50 mM Tris HCl (pH 8.6) was added to dissolve the sample mixture. This was followed by addition of 4 μl of a 4 mM aqueous solution of TCEP (Tris(2-carboxyethyl)phosphine hydrochloride) and the solution was incubated at 37° C. for 30 minutes. Subsequently, 1 μl of a 500 mM aqueous solution of iodoacetamide was added and the mixture was incubated in a dark place at room temperature for 45 minutes.

To the sample mixture, 450 μl of an aqueous solution of 5 mM CaCl₂/50 mM Tris-HCl (pH 7.8) dissolved 2 μg of trypsin therein was added and the mixture was incubated at 37° C. for 16 hours to carry out enzymatic digestion.

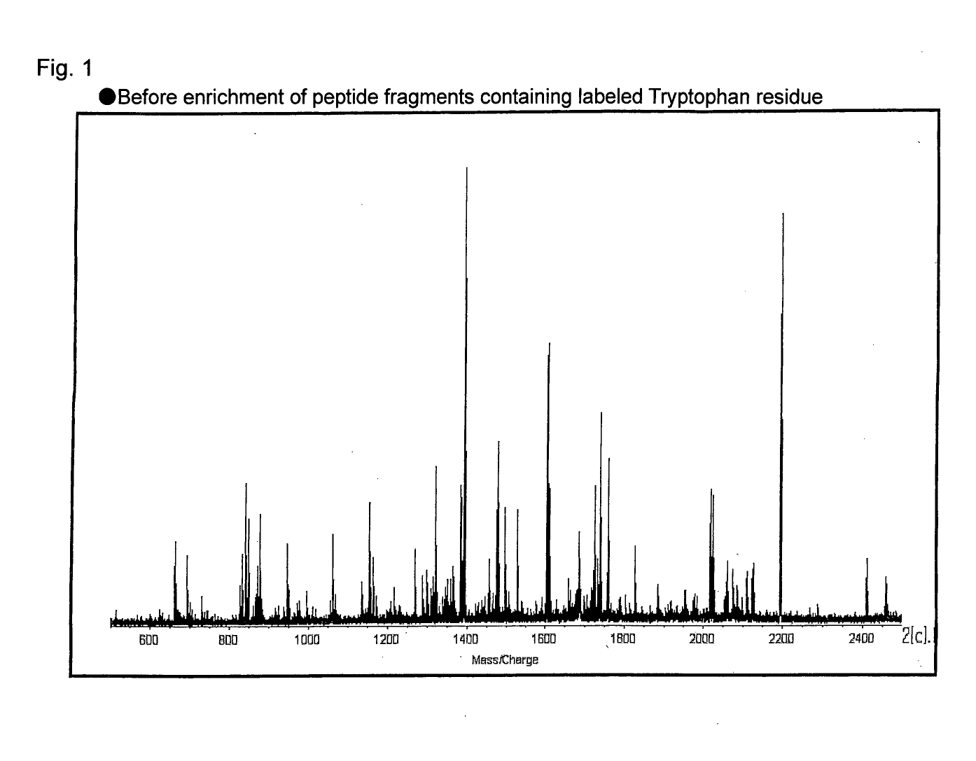

Using desalting tips (ZipTip C18, Millipore), the treated sample mixture was desalted and was then subjected to MALDI-TOF MS analysis. The resulting spectrum is shown in FIG. 1 entitled “Before the enrichment of peptide fragments containing labeled tryptophan residues”. In FIG. 1, horizontal axis represents the mass/charge ratio and vertical axis represents the intensity of the fragment ions.

(b) Enrichment of Peptides Containing Labeled Tryptophan

YMC-Pack Ph packing material (YMC Corp.) was allowed to swell in an aqueous solution of 50 mM KH₂PO₄/CH₃CN/MeOH=68/6/26 overnight. The swollen YMC-PACK ph slurry gel was packed to a volume of 1 ml.

The sample mixture obtained in the procedure (a), after dried up in centrifugal evaporator, was redissolved in 100 μl of a wash buffer (aqueous solution of 50 mM KH₂PO₄/CH₃CN/MeOH =68/6/26). The resulting sample solution was applied to the column (phenyl column) prepared in the procedure (b) above. The wash buffer was used as the mobile phase. Using a syringe, the flow rate was adjusted to 6 droplets/min (1 droplet=approx. 25 μl). Each fraction was collected in a volume of 500 μl. Under this condition, a total of 12 fractions were collected. In this manner, the fractionation using the wash buffer as the mobile phase gave twelve fractions (i.e., Wash fractions (1) to (12)).

The mobile phase was then switched to an elute buffer (aqueous solution of 50 mM KH₂PO₄/CH₃CN/MeOH=4/18/78) and was passed through the column to elute the peptides containing labeled tryptophan. The elution was carried out at a flow rate of 6 droplets/min (1 droplet=approx. 25 μl) with each fraction collected in a volume of 500 μl. A total of five elute fractions, each including peptides containing labeled tryptophan, were collected (Elute fractions (1) to (5)) through the described elution process with the elute buffer.

Each of the fractions obtained through the successive fractionation with the wash buffer and the elute buffer was dried up in a centrifugal evaporator. This dried sample was redissolved in 100 μl of a 0.1% aqueous TFA (trifluoroacetic acid) solution and the solution was desalted with desalting tips (ZipTipμC18, Millipore).

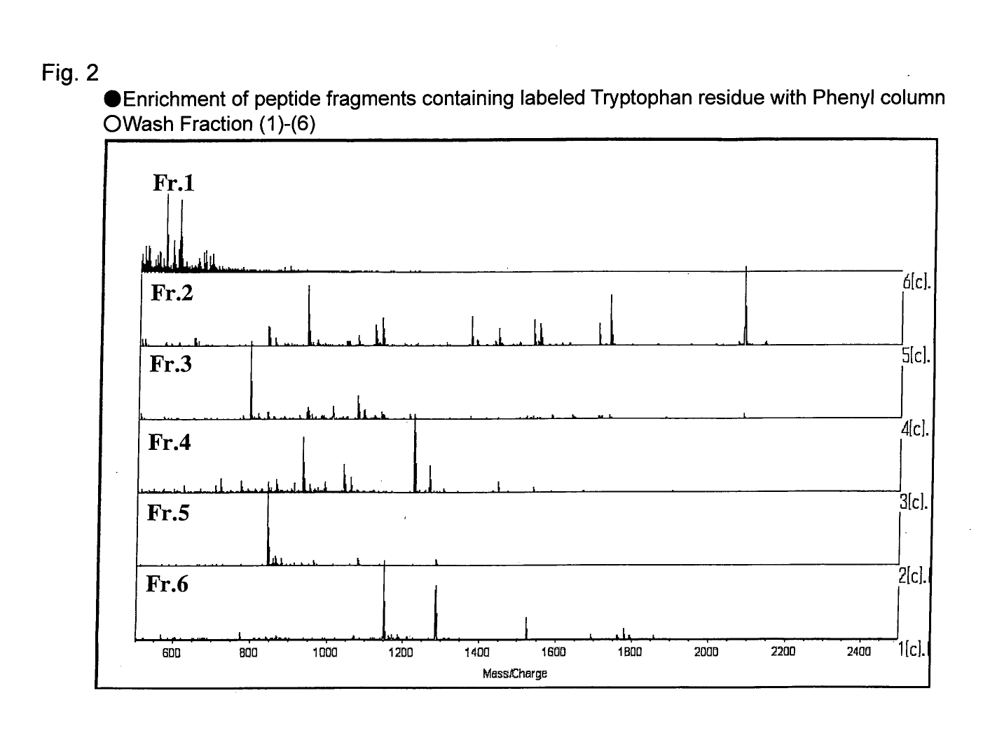

The fraction samples thus obtained were each analyzed with MALDI-TOF MS, and the resulting mass spectra were shown in FIGS. 2 to 4 (entitled “Enrichment of peptide fragments containing labeled tryptophan residue with phenyl column”). In each figure, horizontal axis represents the mass/charge ratio and vertical axis represents the intensity of fragment ions.

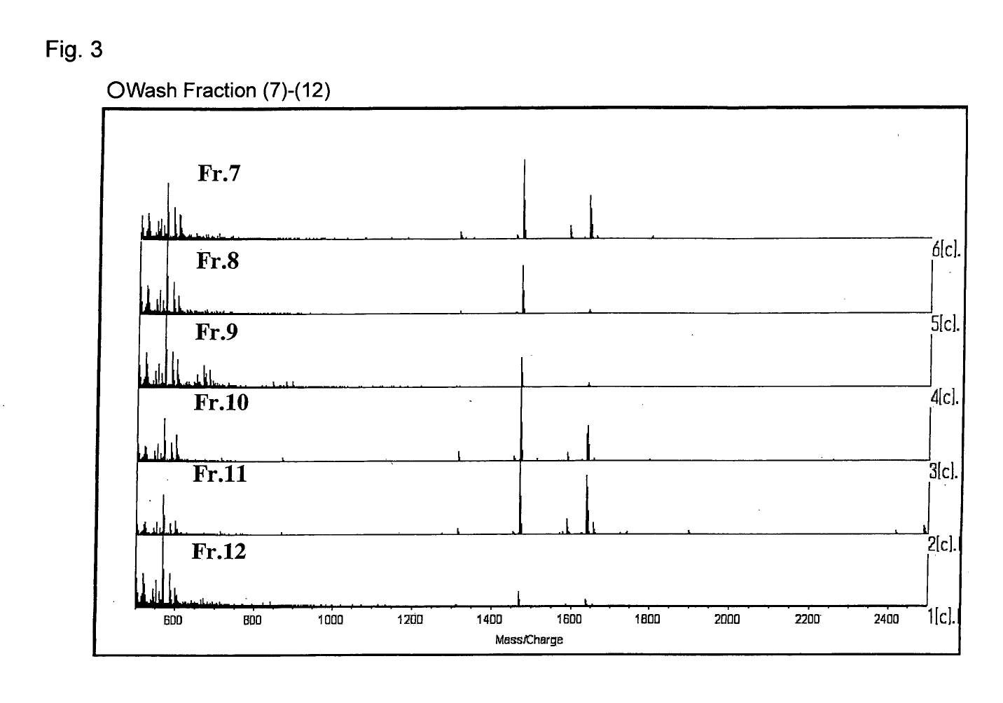

Of the twelve fractions obtained by the washing with the wash buffer, the spectra for the first fraction (Fr. 1) through the sixth fraction (Fr. 6) (Wash fractions (1) through (6)) are shown in FIG. 2, and the spectra for the seventh fraction (Fr. 7) through the twelfth fraction (Fr. 12) (Wash fractions (7) through (12)) are shown in FIG. 3.

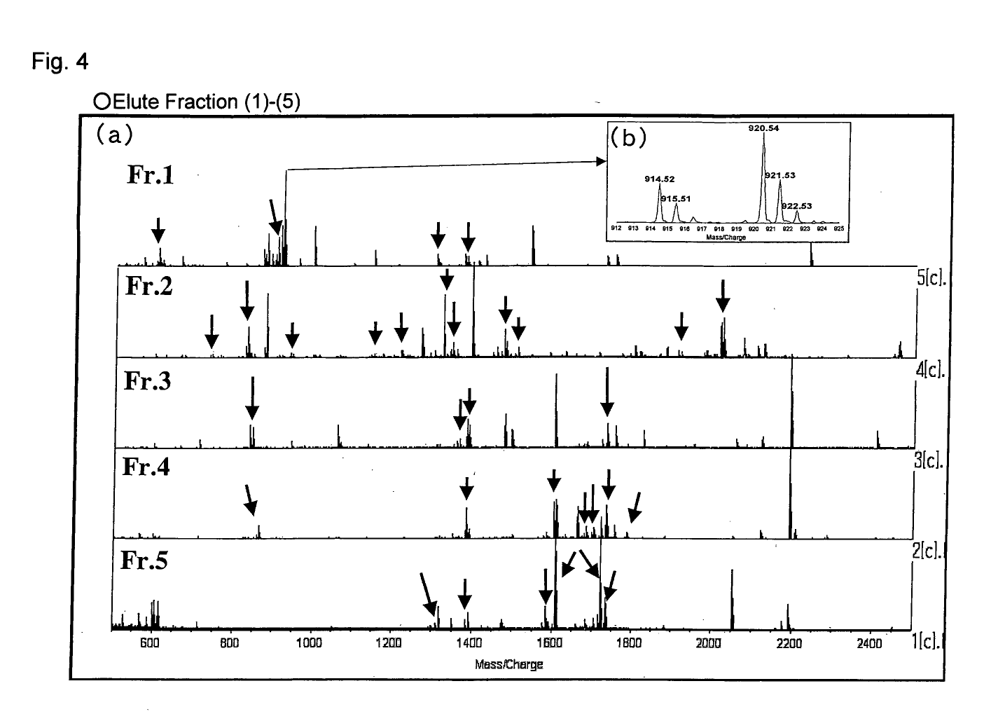

FIG. 4-a shows the spectra for the first fraction (Fr. 1) through the fifth fraction (Fr. 5) (Elute fractions (1) through (5)) obtained by the elution with the elute buffer. In FIG. 4-a, arrows indicate paired peaks for peptide fragments containing NBSCl-labeled tryptophan. One of the pairs is expanded and is shown in FIG. 4-b. As shown in FIG. 4-b, the paired peaks has 6 daltons mass difference which corresponds to the mass difference between the peptide fragment containing (¹²C) NBSCl-labeled tryptophan and the same peptide fragment containing (¹³C) NBSCl-labeled tryptophan. The paired peaks also have an area ratio of 1:2, which corresponds to the mixed ratio of the labeled and non-labeled peptides. These paired peaks are detected only in Elute fractions (1) through (5).

These figures indicate that the desired peptide fragments containing labeled tryptophan residues can be eluted to the Elute fraction in a selective manner by using the method of the present invention.

In the Example described above, the peptide containing labeled tryptophan is prepared as the protein or peptide containing amino acid residue with π electron-containing modifying group, by modifying rat serum with nitrobenzenesulfenyl chloride; separated by using the media with phenyl group as the π electron-containing group; and used for mass spectrometry analysis. The present invention, however, is not limited to the above-described peptide, but may be applied to any protein and peptide containing π electron-containing group or π electron-containing modifying group. Also, the present invention is not limited to the above-described media, but may be used any media with π electron-containing group. Therefore, the described Example is intended to be only illustration and should not be construed in any way as limiting the scope of the invention. The invention is intended to cover all changes, modifications and equivalents that may be included within the spirit and the scope of the invention as defined by the appended claims.

1. A method for enrichment/separation of a protein or a peptide, comprising separating a protein or a peptide containing an amino acid residue with a π electron-containing group by using a media with a π electron-containing group.

2. The method according to claim 1, wherein the amino acid residue with a π electron-containing group is tryptophan residue.

3. The method according to claim 1, wherein the π electron-containing group of the media is phenyl group.

4. A method for enrichment/separation of a protein or a peptide, comprising separating a protein or a peptide containing an amino acid residue with a π electron-containing modifying group, which is modified with a π electron-containing compound, by using a media with a π electron-containing group.

5. The method according to claim 4, wherein the amino acid residue is tryptophan residue.

6. The method according to claim 4, wherein the π electron-containing compound is a sulfenyl compound having π electrons.

7. The method according to claim 6, wherein the sulfenyl compound is 2-nitrobenzene sulfenyl chloride.

8. The method according to claim 4, wherein the π electron-containing group of the media is phenyl group.

9. A method for enrichment/separation of a peptide, comprising the steps of:
fragmenting a protein or a peptide containing an amino acid residue with a π electron-containing group, to obtain a fragmented sample solution which contains a peptide fragment containing the amino acid residue with the π electron-containing group and a peptide fragment with no π electron-containing group; and
exposing the fragmented sample solution to a media with a π electron-containing group, to separate the peptide fragment containing the amino acid residue with the π electron-containing group from the peptide fragment with no π electron-containing group.

10. A method for enrichment/separation of a peptide, comprising the steps of:
modifying a protein or a peptide with a π electron-containing compound to obtain a sample solution which contains a protein or a peptide containing an amino acid residue with a π electron-containing modifying group;
fragmenting the protein or the peptide containing the amino acid residue with the π electron-containing modifying group, to obtain a fragmented sample solution which contains a peptide fragment containing the amino acid residue with the π electron-containing group and a peptide fragment with no π electron groups; and
exposing the fragmented sample solution to a media with a π electron-containing group, to separate the peptide fragment containing the amino acid residue with the π electron-containing group from the peptide fragment with no π electron-containing group.
